(19) United States
(12) Patent Application Publication
(10) Pub. No.: US 2024/0271997 A1
(43) Pub. Date: Aug. 15, 2024

(54) DETECTION SUBSTRATE AND DETECTION DEVICE

(71) Applicants: Beijing BOE Optoelectronics Technology Co., Ltd., Beijing (CN); BOE Technology Group Co., Ltd., Beijing (CN)

(72) Inventors: Gen HUANG, Beijing (CN); Hao YAN, Beijing (CN); Shoujin CAI, Beijing (CN); Cheng LI, Beijing (CN); Lin ZHOU, Beijing (CN); Dexi KONG, Beijing (CN); Zixiao CHEN, Beijing (CN); Jin CHENG, Beijing (CN); Jie ZHANG, Beijing (CN); Song CUI, Beijing (CN); Zhiliang PENG, Beijing (CN)

(21) Appl. No.: 18/022,261

(22) PCT Filed: Apr. 27, 2022
(86) PCT No.: PCT/CN2022/089601

§ 371 Date: Feb. 20, 2023

(51) Int. Cl. G01J 1/44; A61B 5/1172; G01T 1/24

(52) U.S. Cl. CPC G01J 1/44; A61B 5/1172; G01J 2001/448; G01T 1/24

(57) ABSTRACT

Disclosed are a detection substrate and a detection device. The detection substrate includes: a plurality of photoelectric converters arranged in an array, where the photoelectric converter includes a plurality of film layers, an area of an overlapping region between electrode film layers forming an internal capacitor in the photoelectric converter is less than an area of the other film layer in the photoelectric converter; and a drive circuit electrically connected to the photoelectric converters.

### CROSS-REFERENCE TO RELATED APPLICATIONS

This application is a National Stage of International Application No. PCT/CN2022/089601, filed Apr. 27, 2022, which is hereby incorporated by reference in its entirety.

### FIELD

The present disclosure relates to the technical field of detection, and particularly to a detection substrate and a detection device.

### BACKGROUND

In the fields of medical X-ray imaging and fingerprint identification, an amorphous silicon (a-Si) based thin film transistor (TFT) technology has been extensively applied. In order to improve performances (for example, a noise reduction performance and a signal amplification capability enhancement performance) of a thin film transistor, an active pixel sensor structure is generally adopted for constituting a detection panel for X-ray or fingerprint identification.

The APS structure includes three TFTs and one positive-intrinsic-negative (PIN) photodiode. Generally, an amplification gain of the APS structure is inversely proportional to PIN capacitance of the photodiode. Accordingly, low PIN capacitance will generate considerable change in signal voltage, higher sensitivity, lower dosage and higher image quality. A smaller-sized PIN capacitor is required for attaining low PIN capacitance, which reduces a lighting area of the PIN capacitor and a light entry amount instead.

### SUMMARY

In a first aspect, in order to solve the above technical problem, an embodiment of the present disclosure provides a detection substrate. The detection substrate includes: a plurality of photoelectric converters arranged in an array, where the photoelectric converter includes a plurality of film layers, an area of an overlapping region between electrode film layers forming an internal capacitor in the photoelectric converter is less than an area of the other film layer in the photoelectric converter; and a drive circuit electrically connected to the photoelectric converters.

In a possible implementation mode, the photoelectric converter includes: a first electrode layer, a first semiconductor layer, an intrinsic semiconductor layer, a second semiconductor layer and a second electrode layer that are sequentially stacked: the first semiconductor layer and the second semiconductor layer are different heavily doped semiconductor layers; and one electrode film layer of the internal capacitor is composed of the first electrode layer and the first semiconductor layer, and the other electrode film layer of the internal capacitor is composed of the second electrode layer and the second semiconductor laver.

In a possible implementation mode, the first semiconductor layer and the second semiconductor layer are doped with different elements.

In a possible implementation mode, an area of an orthographic projection of the second electrode layer and the second semiconductor layer on the intrinsic semiconductor layer is greater than and completely covers an area of an orthographic projection of the first electrode layer and the first semiconductor layer on the intrinsic semiconductor layer: or an area of an orthographic projection of the first electrode layer and the first semiconductor layer on the intrinsic semiconductor layer is greater than and completely covers an area of an orthographic projection of the second electrode layer and the second semiconductor layer on the intrinsic semiconductor layer.

In a possible implementation mode, the second electrode layer and the second semiconductor layer have patterns that coincide with each other; and the first electrode layer and the first semiconductor layer have patterns that coincide with each other.

In a possible implementation mode, a pattern of the overlapping region includes a symmetrical pattern.

In a possible implementation mode, the symmetrical pattern is a cross, a center of the cross substantially coincides with a center of the intrinsic semiconductor layer, and the cross extends to an edge of the intrinsic semiconductor layer.

In a possible implementation mode, the symmetrical pattern is a ring, the ring is consistent with a shape of an outer contour of the intrinsic semiconductor layer, and the ring is arranged within the outer contour of the intrinsic semiconductor layer and keeps a certain distance from an outer edge of the intrinsic semiconductor layer.

In a possible implementation mode, the symmetrical pattern is a plurality of rectangles arranged in an array, and the symmetrical pattern is arranged at corners of the intrinsic semiconductor layer.

In a possible implementation mode, the symmetrical pattern is a circle or a rectangle, and a center of the circle or the rectangle substantially coincides with a center of the intrinsic semiconductor layer.

In a possible implementation mode, the symmetrical pattern is a plurality of strips arranged at intervals, and the strips extend to an edge of the intrinsic semiconductor layer.

In a possible implementation mode, the symmetrical pattern is a grid, the grid is composed of a plurality of strips that cross each other, and each strip extends to an edge of the intrinsic semiconductor layer.

In a possible implementation mode, the symmetrical pattern is a plane having a plurality of circular hollow patterns arranged in an array.

In a possible implementation mode, the area of the overlapping region between the electrode film layers of the internal capacitor of each photoelectric converter is the same.

In a possible implementation mode, the detection substrate further includes: a plurality of microlenses corresponding to the photoelectric converters in a one-to-one manner and arranged on light entry sides of the photoelectric converters.

In a second aspect, an embodiment of the present disclosure provides a detection device. The detection device includes the detection substrate as described in the first aspect.

### BRIEF DESCRIPTION OF THE DRAWINGS

FIG. 1 is a schematic structural diagram of a detection substrate according to an embodiment of the present disclosure;

FIG. 2 is a schematic diagram of an overlapping area between electrode film layers forming an internal capacitor of a photoelectric converter according to an embodiment of the present disclosure;

FIGS. 3 and 4 are schematic structural diagrams of photoelectric converters according to embodiments of the present disclosure;

FIGS. 5 and 6 are schematic diagrams of orthographic projections of second electrode layers and second semiconductor layers on intrinsic semiconductor layers according to embodiments of the present disclosure;

FIG. 7 is a positional relation diagram of ring-shaped overlapping regions and intrinsic semiconductor layers according to embodiments of the present disclosure;

FIG. 8 is another positional relation diagram of ring-shaped overlapping regions and intrinsic semiconductor layers according to embodiments of the present disclosure;

FIG. 9 is another positional relation diagram of ring-shaped overlapping regions and intrinsic semiconductor layers according to embodiments of the present disclosure;

FIG. 10 is a positional relation diagram of a plurality of rectangular overlapping regions arranged in an array and intrinsic semiconductor layers according to embodiments of the present disclosure;

FIG. 11 is another positional relation diagram of a plurality of rectangular overlapping regions arranged in an array and intrinsic semiconductor layers according to embodiments of the present disclosure;

FIG. 12 is a positional relation diagram of a plurality of circular overlapping regions arranged in an array and an intrinsic semiconductor layer according to an embodiment of the present disclosure;

FIG. 13 is a positional relation diagram of a circular overlapping region and an intrinsic semiconductor layer according to an embodiment of the present disclosure;

FIG. 14 is a positional relation diagram of a rectangular overlapping region and an intrinsic semiconductor layer according to an embodiment of the present disclosure;

FIG. 15 is a positional relation diagram of a plurality of strip-shaped overlapping regions arranged at intervals and an intrinsic semiconductor layer according to an embodiment of the present disclosure;

FIG. 16 is a positional relation diagram of grid-shaped overlapping regions and an intrinsic semiconductor layer according to an embodiment of the present disclosure;

FIG. 17 is a positional relation diagram of planar overlapping regions and an intrinsic semiconductor layer according to an embodiment of the present disclosure;

FIG. 18 is a schematic composition diagram of detection units according to the present disclosure;

FIG. 19 is another schematic composition diagram of detection units according to the present disclosure;

FIG. 20 is a schematic partial view of detection substrates according to embodiments of the present disclosure;

FIG. 21 is another schematic partial view of detection substrates according to embodiments of the present disclosure;

FIG. 22 is a schematic diagram of a drive circuit of a photoelectric converter according to an embodiment of the present disclosure.

### DETAILED DESCRIPTION OF THE EMBODIMENTS

In the related art, the light entry amount is increased through a microlens light-collecting solution, which requires a microlens process, thereby increasing production cost. Since a microlens itself can reflect or absorb part of light energy, energy loss is caused. Moreover, a final imaging effect will be poor due to inferior quality of a microlens.

In view of this, how to increase an amplification gain of an APS structure without reducing a light entry amount has become an urgent technical problem.

Embodiments of the present disclosure provide a detection substrate and a detection device, which are used for solving the problem that if an amplification gain of an active pixel sensor (APS) structure is increased, a light entry amount will be reduced in the related art.

In order to make the above objective, features and advantages of the present disclosure clearer and more comprehensible, the present disclosure will be further described below in combination with accompanying drawings and embodiments. However, exemplary implementation modes can be implemented in many forms and should not be construed as being limited to implementation modes set forth herein. On the contrary, these implementation modes are provided to make the present disclosure more thorough and complete, and to fully convey the concept of exemplary implementation modes to those skilled in the art. In patterns, the same reference numerals denote the same or similar structures, and thus the repeated description thereof will be omitted. Words expressing positions and directions described in the present disclosure are all described by taking patterns as examples, but changes can be made according to requirements, and the made changes all fall within the scope of protection of the present disclosure. Accompanying drawings of the present disclosure are only used to illustrate relative positional relations but do not represent the true scale.

It should be noted that specific details are set forth in the following description to facilitate full understanding of the present disclosure. However, the present disclosure can be implemented in many other ways different from those described herein, similar improvements can be made by those skilled in the art without departing from the connotation of the present disclosure. Therefore, the present disclosure is not limited by particular implementation modes disclosed below: Subsequent illustration of the description describes preferred implementation modes for implementing the present disclosure. However, the illustration is used for the purpose of illustrating the general principles of the present disclosure and is not intended to limit the scope of the present disclosure. The scope of protection of the present disclosure should be subject to the appended claims.

A detection substrate and a detection device provided in embodiments of the present disclosure will be described in detail below in combination with accompanying drawings.

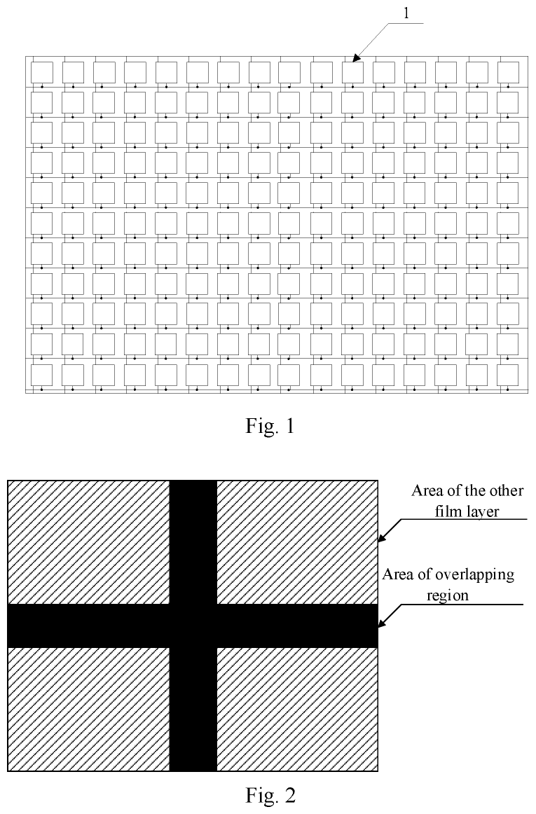

With reference to FIG. 1, a schematic structural diagram of a detection substrate according to an embodiment of the present disclosure is shown. The detection substrate includes:
a plurality of photoelectric converters 1 arranged in an array, where the photoelectric converter 1 includes a plurality of film layers, and an area of an overlapping region between electrode film layers forming an internal capacitor in the photoelectric converter 1 is less than an area of other film layers in the photoelectric converter 1; and the area of the above overlapping region may be determined according to minimum design capacitance of the internal capacitor, for example, under the condition that the minimum design capacitance of the internal capacitor is 0.162 pF, the area of the electrode overlapping region of the internal capacitor corresponding to the minimum design capacitance is 1574 um²; and
a drive circuit (not shown in FIG. 1) electrically connected to the photoelectric converters 1.

With reference to FIG. 2, a schematic diagram of an overlapping area of electrode film layers forming an internal capacitor of a photoelectric converter according to an embodiment of the present disclosure is shown. FIG. 2 is a top view of the photoelectric converter 1. In FIG. 2, a black region is the area of the overlapping region between the electrode film layers forming the internal capacitor in the photoelectric converter, and an oblique line region (including the black region) is the area of the other film layer.

In an embodiment provided in the present disclosure, by setting the area of the overlapping region between the electrode film layers forming the internal capacitor in the photoelectric converter 1 to be less than the area of the other film layer in the photoelectric converter 1, the area of the other film layer may keep large to keep a large light entry amount while internal capacitance of the photoelectric converter 1 is reduced, such that a large light entry amount of the photoelectric converter may be kept while an amplification gain of the drive circuit connected to the photoelectric converter 1 is increased, thereby improving sensitivity of the detection substrate and image quality. If the above detection substrate is an X-ray detection substrate, the requirement on a dosage of a related reagent may be reduced.

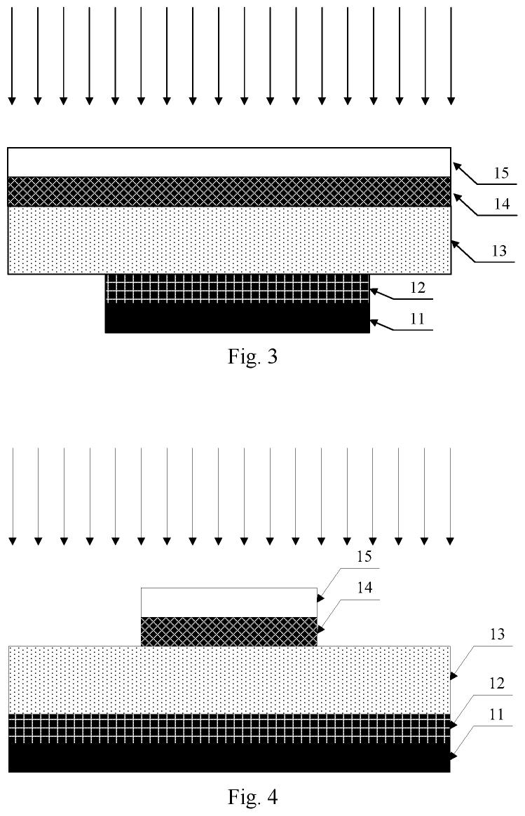

With reference to FIGS. 3 and 4, schematic structural diagrams of photoelectric converters according to embodiments of the present disclosure are shown. The photoelectric converter includes:
a first electrode layer 11, a first semiconductor layer 12, an intrinsic semiconductor layer 13, a second semiconductor layer 14 and a second electrode layer 15 that are sequentially stacked. The first semiconductor layer 12 and the second semiconductor layer 14 are different heavily doped semiconductor layers. The first semiconductor layer 12, the intrinsic semiconductor layer 13 and the second semiconductor layer 14 are all made of amorphous silicon materials. The difference is that the first semiconductor layer 12 and the second semiconductor layer 13 are made of amorphous silicon materials doped with different elements, but the intrinsic semiconductor layer 13 is made of an undoped amorphous silicon material.

An electrode corresponding to a light entry side of the photoelectric converter 1 is a transparent electrode. If light enters the photoelectric converter 1 from the second electrode layer 15, the second electrode layer 15 is a transparent electrode. After penetrating the transparent electrode, the light is required to penetrate the second semiconductor layer 14 (for example, a P-doped semiconductor layer) having a certain thickness to reach the intrinsic semiconductor layer 13 to be effectively converted into a photocurrent. Moreover, the second semiconductor layer 14 absorbs light without making any contributions to external quantum efficiency (EQE), and an absorption coefficient of the amorphous silicon material to visible light is close to 10⁶ cm⁻¹ (especially for short waves), such that by reducing the area of the second doped semiconductor layer 14, more light may reach a depletion region of the photoelectric converter, and a short wave response is enhanced.

One electrode film layer of the internal capacitor is composed of the first electrode layer 11 and the first semiconductor layer 12, and the other electrode film layer of the internal capacitor is composed of the second electrode layer 15 and the second semiconductor layer 14. The first semiconductor layer 12 and the second semiconductor layer 14 are doped with different elements. For example, if the first semiconductor film layer is N-type doped semiconductor film layer (which may be doped with pentavalent impurity elements such as phosphorus and arsenic, and phosphorus ions are used herein), the second semiconductor film layer is a P-type doped semiconductor film layer (which may be doped with trivalent impurity elements such as boron and gallium, and boron ions are used herein). If the first semiconductor film layer is a P-type doped semiconductor film layer, the second semiconductor layer is an N-type doped semiconductor film layer.

In some embodiments, an area of an orthographic projection of the second electrode layer 15 and the second semiconductor layer 14 on the intrinsic semiconductor layer 13 is greater than and completely covers an area of an orthographic projection of the first electrode layer 11 and the first semiconductor layer 12 on the intrinsic semiconductor layer 13.

As shown in FIG. 3, an area of the electrode film layer formed by the second electrode layer 15 and the second semiconductor layer 14 (that is, the area of the orthographic projection of the second electrode layer 15 and the second semiconductor layer 14 on the intrinsic semiconductor layer 13) is greater than and completely covers an area of the electrode film layer formed by the first electrode layer 11 and the first semiconductor layer 12 (that is, the area of the orthographic projection of the first electrode layer 11 and the first semiconductor layer 12 on the intrinsic semiconductor layer 13).

In some other embodiments, the area of the orthographic projection of the first electrode layer 11 and the first semiconductor layer 12 on the intrinsic semiconductor layer 13 is greater than and completely covers the area of the orthographic projection of the second electrode layer 15 and the second semiconductor layer 14 on the intrinsic semiconductor layer 13. As shown in FIG. 4, an area of the electrode film layer formed by the first electrode layer 11 and the first semiconductor layer 12 is greater than and completely covers an area of the electrode film layer formed by the second electrode layer 15 and the second semiconductor layer 14.

In an embodiment provided in the present disclosure, the first semiconductor film layer 12 having an electric conduction property and the first electrode layer 11, and the second semiconductor film layer 14 having an electric conduction property and the second electrode layer 15 are regarded as two electrode film layers of the internal capacitor of the photoelectric converter 1, and the area of the electrode film layer formed by the second electrode layer 15 and the second semiconductor layer 14 is greater than and completely covers the area of the electrode film layer formed by the first electrode layer 11 and the first semiconductor layer 12; or the area of the electrode film layer formed by the first electrode layer 11 and the first semiconductor layer 12 is greater than and completely covers the area of the electrode film layer formed by the second electrode layer 15 and the second semiconductor layer 14, and an overlapping area of the electrode film layers forming the internal capacitor of the photoelectric converter 1 is less than an area of the other film layer (that is, the intrinsic semiconductor layer) in the photoelectric converter 1. Therefore, internal capacitance of the photoelectric converter 1 is reduced, and a light entry amount of the photoelectric converter 1 keeps unchanged.

As shown in FIG. 4, by setting an area of the second electrode layer 15 and a second intrinsic semiconductor layer 13 that are stacked on the intrinsic semiconductor layer 13 to be less than an area of the intrinsic semiconductor layer 13, an original area of the first semiconductor layer 12 and the second semiconductor layer 14 under the intrinsic semiconductor layer 13 may be kept, and the film layers may be conveniently deposited when the photoelectric converter 1 is manufactured.

In some embodiments, film layers forming the same electrode film layer of the internal capacitor of the photoelectric converter 1 may have patterns that coincide with each other. As shown in FIGS. 3 and 4, the second electrode layer 15 and the second semiconductor layer 14 form the same electrode film layer of the internal capacitor of the photoelectric converter 1, and the second electrode layer 15 and the second semiconductor layer 14 have patterns that coincide with each other. The first electrode layer 11 and the first semiconductor layer 12 form the other electrode film layer of the internal capacitor of the photoelectric converter 1, and the first electrode layer 11 and the first semiconductor layer 12 have patterns that coincide with each other.

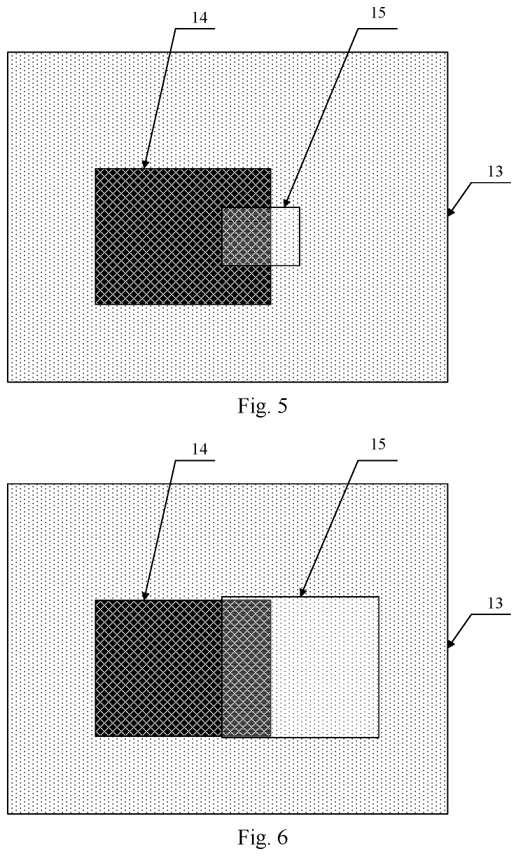

In other embodiments, film layers forming the same electrode film layer of the internal capacitor of the photoelectric converter 1 may have different patterns that partially coincide with each other, or have the same patterns that partially coincide with each other. With reference to FIGS. 5 and 6, schematic diagrams of orthographic projections of second electrode layers and second semiconductor layers on intrinsic semiconductor layers according to embodiments of the present disclosure are shown. As shown in FIG. 5, the second electrode layer 15 and the second semiconductor layer 14 form the same electrode film layer of the internal capacitor of the photoelectric converter 1, and the second electrode layer 15 and the second semiconductor layer 14 have different patterns that partially coincide with each other. As shown in FIG. 6, the second electrode layer 15 and the second semiconductor layer 14 form the same electrode film layer of the internal capacitor of the photoelectric converter 1, and the second electrode layer 15 and the second semiconductor layer 14 have the same patterns that partially coincide with each other. In FIGS. 5 and 6, an area of an orthographic projection of the electrode film layer of the internal capacitor formed by the second electrode layer 15 and the second semiconductor layer 14 is an area of a pattern formed by the second electrode layer 15 and the second semiconductor layer 14. Similarly, the first electrode layer 11 and the first semiconductor layer 12 forming the same electrode film layer of the internal capacitor of the photoelectric converter 1 may have different patterns that partially coincide with each other, or have the same patterns that partially coincide with each other, which will not be described in detail herein.

In some embodiments, a pattern of an overlapping region between the electrode film layers forming the internal capacitor in the photoelectric converter 1 includes a symmetrical pattern, that is, an overlapping region of an orthographic projection of the first electrode layer 11 and the first semiconductor layer 12 on the intrinsic semiconductor layer 13 and an orthographic projection of the second electrode layer 15 and the second semiconductor layer 14 on the intrinsic semiconductor layer 13 may be a symmetrical pattern, and the symmetrical pattern may be at least one or a combination of a circle, a square, a ring, a cross, a grid, a strip and a rectangle. The minimum size of the above symmetrical pattern is the accuracy of a used lithography device or etching device, for example, under the condition that the accuracy of the lithography device reaches 14 um, the minimum size of the above symmetrical pattern may reach 14 um.

In some embodiments, the symmetrical pattern may be a cross, a center of the cross substantially coincides with a center of the intrinsic semiconductor layer 13, and the cross extends to an edge of the intrinsic semiconductor layer 13.

As shown in FIG. 2, the pattern of the overlapping region between the electrode film layers forming the internal capacitor in the photoelectric converter 1 is a cross as shown by the black region in FIG. 2, a center of the cross substantially coincides with a center of an intrinsic semiconductor layer 13 (that is, the other film layer in FIG. 2), and the cross extends to an edge of the intrinsic semiconductor layer 13, such that an electric field in the internal capacitor of the photoelectric converter 1 may be uniformly distributed.

In some other embodiments, the center of the above cross may deviate from the center of the intrinsic semiconductor layer 13, and an edge of the above cross may keep a certain distance from the edge of the intrinsic semiconductor layer 3.

In some embodiments, the symmetrical pattern may be a ring, the ring is consistent with a shape of an outer contour of the intrinsic semiconductor layer 13, and the ring is arranged within the outer contour of the intrinsic semiconductor layer 13 and keeps a certain distance from an outer edge of the intrinsic semiconductor layer 13.

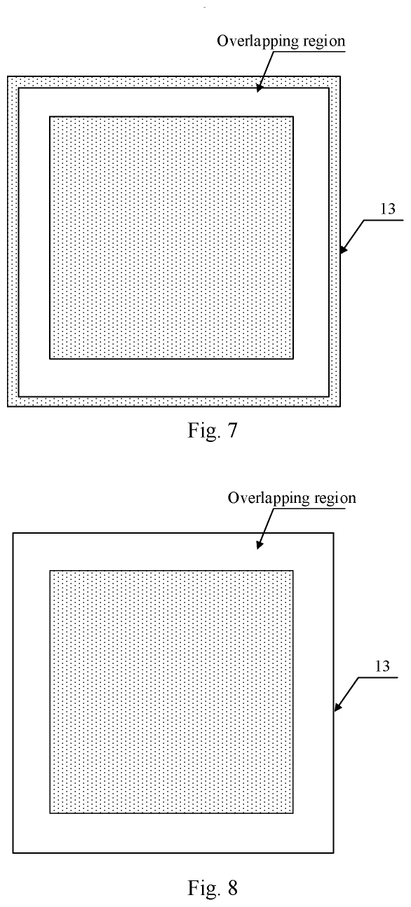

With reference to FIGS. 7-9, positional relation diagrams of ring-shaped overlapping regions and intrinsic semiconductor layers according to embodiments of the present disclosure are shown. In FIG. 7, an outer contour of the intrinsic semiconductor layer (the outermost line in FIG. 7) is a square, such that the pattern of the overlapping region between the electrode film layers forming the internal capacitor in the photoelectric converter 1 is a square ring as shown by a white region in FIG. 7. The above ring is located within the outer contour of the intrinsic semiconductor layer 13, an outer edge of the ring keeps a certain distance from the outer contour of the intrinsic semiconductor layer 13, and a center of the above ring is substantially the same as a center of the intrinsic semiconductor layer 13. Therefore, the overlapping region between the electrode film layers forming the internal capacitor of the photoelectric converter 1 may be reduced, and an electric field in the internal capacitor of the photoelectric converter 1 may be uniformly distributed.

In some other embodiments, the outer edge of the above ring may coincide with the outer contour of the intrinsic semiconductor layer 13, which is as shown in FIG. 8.

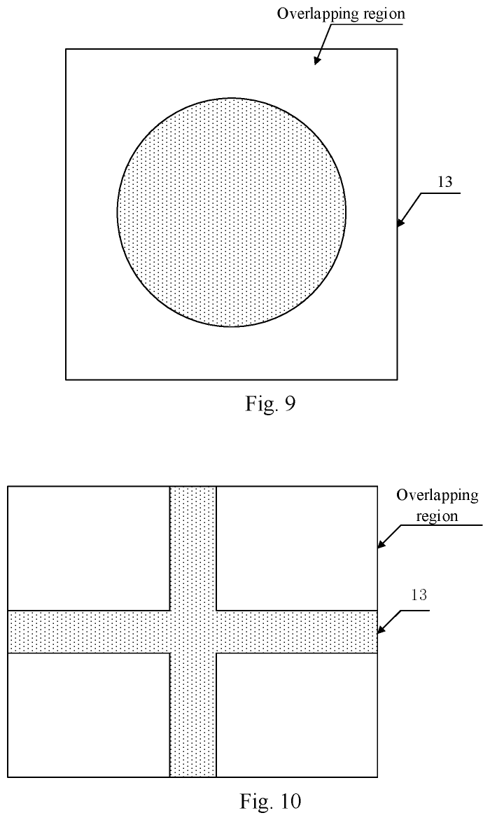

In some other embodiments, a shape of an inner edge and a shape of an outer edge of the above ring may be the same, as shown in FIGS. 7 and 8; the shape of the inner edge and the shape of the outer edge of the ring may be different, as shown in FIG. 9; and in FIG. 9, the outer edge of the ring coincides with the outer contour of the intrinsic semiconductor layer 13.

It should be understood that since central regions of the rings shown in FIGS. 7-9 are hollow (regions within inner contours of the rings are hollow), part of the intrinsic semiconductor layers 13 may be observed through corresponding hollow regions, and it should not be understood that regions of the intrinsic semiconductor layers 13 are only limited to the hollow regions.

In some embodiments, the symmetrical pattern is a plurality of rectangles or circles arranged in an array, and the symmetrical pattern is arranged at corners of the intrinsic semiconductor layer.

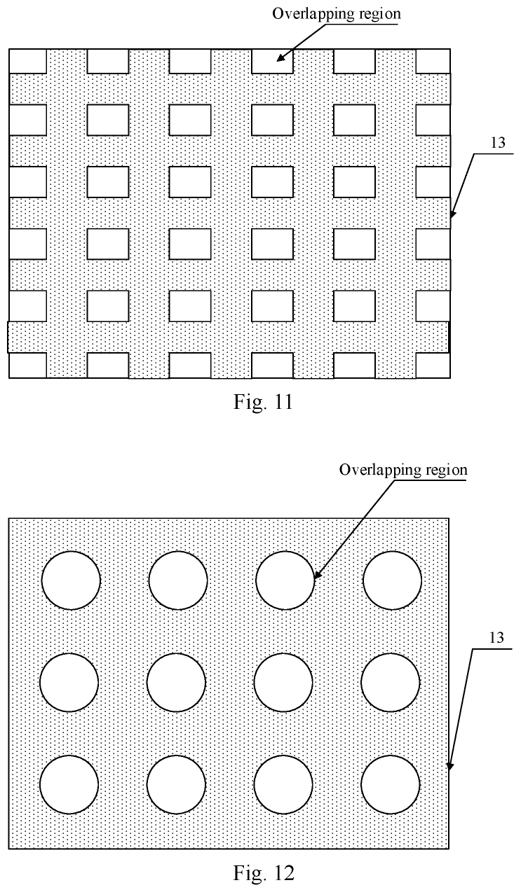

With reference to FIGS. 10 and 11, positional relation diagrams of a plurality of rectangular overlapping regions arranged in an array and intrinsic semiconductor layers according to embodiments of the present disclosure are shown. In FIG. 10, the intrinsic semiconductor layer 13 is a large rectangle, the overlapping region between the electrode film layers of the internal capacitor of the photoelectric converter 1 is a plurality of rectangles (four white regions in FIG. 10) arranged in an array, and these rectangles are arranged at four corners of the intrinsic semiconductor layer 13. Two edges of the above rectangles arranged in an array close to the corners of the intrinsic semiconductor layer 13 may coincide with two edges of the corresponding corners of the intrinsic semiconductor layer 13, or may keep a certain distance from two edges of the corresponding corners of the intrinsic semiconductor layer 13. In this way, the intrinsic semiconductor layer 13 of the photoelectric converter 1 may be completely covered with an electric field of the internal capacitor of the photoelectric converter 1.

As shown in FIG. 11, the rectangles (white regions in FIG. 11) arranged in an array may be set to be less such that the overlapping region may be more uniformly distributed within an outer edge of the intrinsic semiconductor layer 13. Moreover, sides of the rectangles close to the outer edge of the intrinsic semiconductor layer 13 partially coincide with a side of the intrinsic semiconductor layer 13, such that the electric field of the internal capacitor of the photoelectric converter 1 may be more uniformly distributed.

With reference to FIG. 12, a positional relation diagram of a plurality of circular overlapping regions arranged in an array and an intrinsic semiconductor layer according to an embodiment of the present disclosure is shown. The overlapping region between the electrode film layers of the internal capacitor of the photoelectric converter 1 is a plurality of circles (white regions in FIG. 12) arranged in an array, which are located within an outer contour of the intrinsic semiconductor layer 13, and edges of the circles close to the intrinsic semiconductor layer 13 have a certain distance from an edge of the intrinsic semiconductor layer 13.

In some embodiments, the symmetrical pattern is a circle, and a center of the circle substantially coincides with a center of the intrinsic semiconductor layer 13.

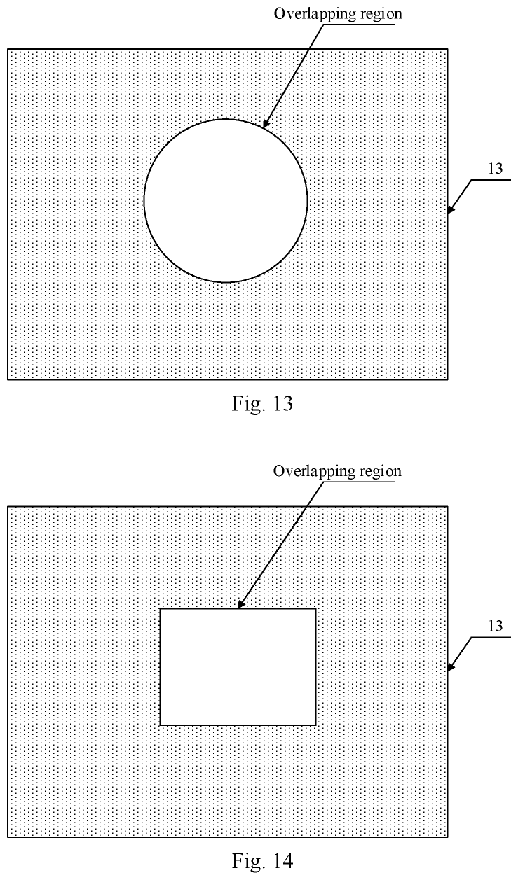

With reference to FIG. 13, a positional relation diagram of circular overlapping region and an intrinsic semiconductor layer according to an embodiment of the present disclosure is shown. The overlapping region between the electrode film layers of the internal capacitor of the photoelectric converter 1 is a circle (white region in FIG. 13), a center of the circle substantially coincides with a center of the intrinsic semiconductor layer 13, the circle is located within an outer contour of the intrinsic semiconductor layer 13, and an edge of the circle keeps a certain distance from an edge of the intrinsic semiconductor layer 13.

In some other embodiments, the center of the above circle may deviate from the center of the intrinsic semiconductor layer 13.

With reference to FIG. 14, a positional relation diagram of a rectangular overlapping region and an intrinsic semiconductor layer according to an embodiment of the present disclosure is shown. The overlapping region between the electrode film layers of the internal capacitor of the photoelectric converter 1 is a rectangle (white region in FIG. 14), a center of the rectangle substantially coincides with a center of the intrinsic semiconductor layer 13, the rectangle is located within an outer contour of the intrinsic semiconductor layer 13, and an edge of the rectangle keeps a certain distance from an edge of the intrinsic semiconductor layer 13.

In some other embodiments, the symmetrical pattern may be a regular polygon, such as a square, a regular pentagon, a regular hexagon and a regular octagon, and positional relations between the regular polygons and intrinsic semiconductor layers 13 are similar to that shown in FIGS. 13 and 14, and will not be described in detail herein.

In some other embodiments, a center of the above circle, rectangle, or regular polygon may deviate from a center of the intrinsic semiconductor layer 13.

In some embodiments, the symmetrical pattern is a plurality of strips arranged at intervals, and the strips extend to an edge of the intrinsic semiconductor layer 13.

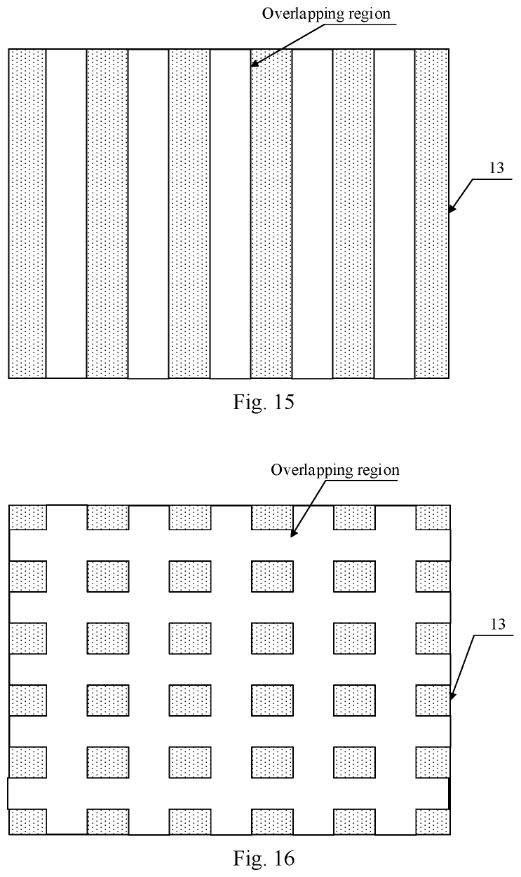

With reference to FIG. 15, a positional relation diagram of a plurality of strip-shaped overlapping regions arranged at intervals and an intrinsic semiconductor layer according to an embodiment of the present disclosure is shown. The overlapping region between the electrode film layers of the internal capacitor of the photoelectric converter 1 is a plurality of strips (white regions in FIG. 15) arranged at intervals, and each strip may extend to an edge of the intrinsic semiconductor layer 13.

In some other embodiments, edges of the strips in FIG. 15 may keep a certain distance from the edge of the intrinsic semiconductor layer 13.

In some embodiments, the symmetrical pattern is a grid, the grid is composed of a plurality of strips that cross each other, and each strip extend to an edge of the intrinsic semiconductor layer.

With reference to FIG. 16, a positional relation diagram of grid-shaped overlapping regions and an intrinsic semiconductor layer according to an embodiment of the present disclosure is shown. The overlapping region between the electrode film layers of the internal capacitor of the photoelectric converter 1 is a grid (white region in FIG. 16) composed of a plurality of strips that cross each other, and each strip may extend to an edge of the intrinsic semiconductor layer 13 such that an electric field of the internal capacitor of the photoelectric converter 1 may be uniformly distributed.

In some other embodiments, edges of the strips forming the grid in FIG. 16 may keep a certain distance from the edge of the intrinsic semiconductor layer 13.

In some embodiments, the symmetrical pattern is a plane having a plurality of circular hollow patterns arranged in an array.

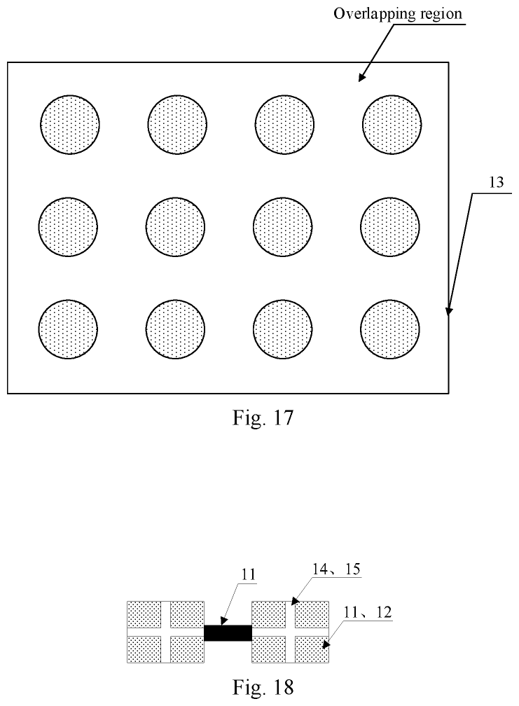

With reference to FIG. 17, a positional relation diagram of planar overlapping regions and an intrinsic semiconductor layer according to an embodiment of the present disclosure is shown. The overlapping region between the electrode film layers of the internal capacitor of the photoelectric converter 1 is a plane (white region in FIG. 17) having a plurality of circular hollow patterns arranged in an array, such that an electric field of the internal capacitor of the photoelectric converter 1 may be uniformly distributed.

In some other embodiments, an edge of the plane in FIG. 17 may extend to an edge of the intrinsic semiconductor layer 13, or may keep a certain distance from the edge of the intrinsic semiconductor layer 13. By simulating a distribution situation of the electric field of the internal capacitor of the photoelectric converter 1, it can be known that there still are fringe fields having certain intensity in uncovered regions of the second electrode layer 15 and the second semiconductor layer 14.

By setting the patterns of the second electrode layer 15 and the second semiconductor layer 14 as combinations of a plurality of shapes (such as a grid, a plurality of stripes, a plane, rectangles or circles arranged in an array, etc.), under the condition of reducing the overlapping area of the electrode film layers of the internal capacitor of the photoelectric converter 1, regions covered by most of non-overlapping regions may also have fringe fields having certain intensity distributed, such that the fringe fields may be used to the maximum extent to increase intensity of the electric field of the internal capacitor, and a response speed of the photoelectric converter 1 may be increased.

In an embodiment provided in the present disclosure, by setting the pattern of the overlapping region of two electrode film layers forming the internal capacitor of the photoelectric converter 1 as a symmetrical pattern, the overlapping area of the electrode film layers forming the internal capacitor of the photoelectric converter 1 may be reduced, thereby reducing the internal capacitance.

In some embodiments, the area of the overlapping region between the electrode film layers of the internal capacitor of the photoelectric converter 1 in the detection substrate is the same such that the photoelectric converter 1 in the detection substrate may have the same detection performance.

In some embodiments, at least two adjacent photoelectric converters 1 form one detection unit and are electrically connected to the same drive circuit; or one photoelectric converter 1 forms one detection unit and is electrically connected to one drive circuit.

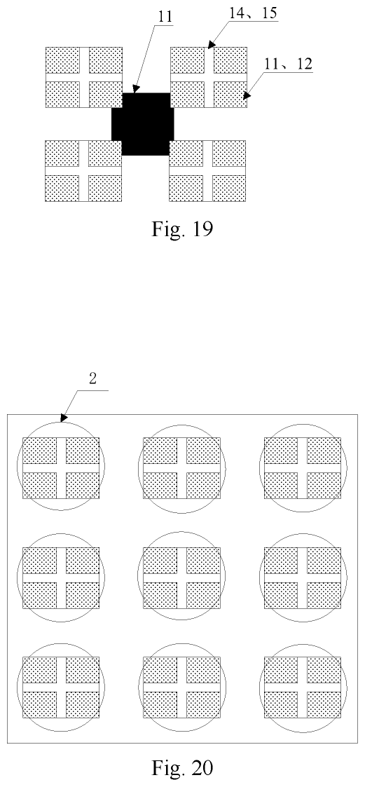

With reference to FIGS. 18 and 19, schematic composition diagrams of detection units according to the present disclosure are shown. In FIG. 18, two photoelectric converters 1 form one detection unit, first electrode layers 11 of the photoelectric converters are electrically connected to each other, and one drive circuit is electrically connected to the detection unit by means of the first electrode layers 11.

In FIG. 19, one detection unit is composed of four photoelectric converters 1, first electrode layers 11 of the photoelectric converters are electrically connected to each other, and one drive circuit is electrically connected to the detection unit by means of the first electrode layers 11.

Certainly, one photoelectric converter 1 may form one detection unit, and the drive circuit is electrically connected to the detection unit by means of a first electrode layer 11.

In an embodiment provided in the present disclosure, by forming one detection unit with a plurality of photoelectric converters 1 and electrically connecting the detection unit to the same drive circuit, capacitance of a single detection unit may be further reduced, a gain of the detection unit may be improved, and sensitivity of the detection unit may be improved.

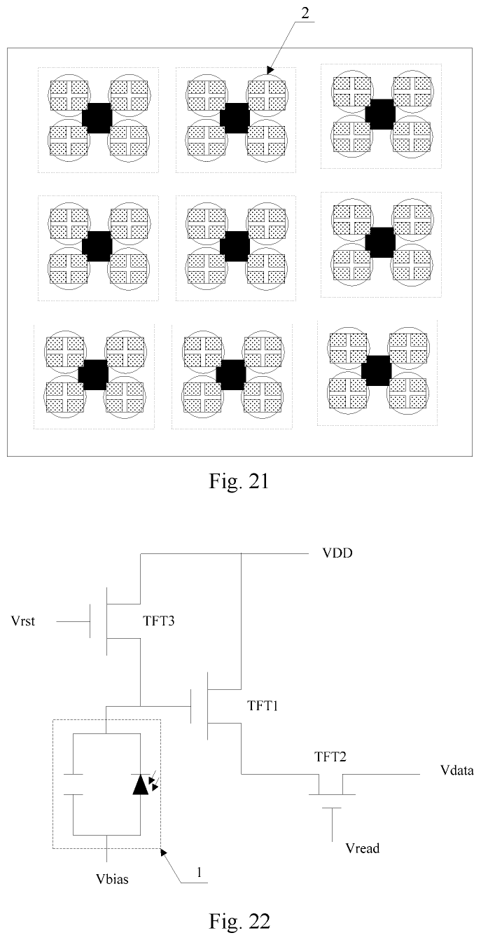

With reference to FIGS. 20 and 21, schematic partial views of detection substrates according to embodiments of the present disclosure are shown. The detection substrate further includes:
a plurality of microlenses 2 corresponding to the photoelectric converters 1 in a one-to-one manner and arranged on light entry sides of the photoelectric converters 1.

In FIGS. 20 and 21, each photoelectric converter 1 has a microlens 2. Assuming that light enters the photoelectric converter 1 from the second electrode layer 15, the corresponding microlens 2 is arranged on one side of the second electrode layer 15 facing away from the first electrode layer 11.

In an embodiment provided in the present disclosure, the corresponding microlens 2 is arranged for each photoelectric converter 1 in the detection substrate, and the microlens 2 are arranged on the light entry sides of the photoelectric converters 1 such that more light may be absorbed into the photoelectric converters 1 through light-collecting and collimating functions of the microlens 2, thereby improving sensitivity of the photoelectric converters 1.

It should be understood that in FIGS. 18-21, orthographic projections of the first electrode layer 11, the first semiconductor layer 12 and the intrinsic semiconductor layer 13 in each photoelectric converter 1 completely coincide with each other, and orthographic projections of the second semiconductor layer 14 and the second electrode layer completely coincide with each other.

With reference to FIG. 22, a schematic diagram of a drive circuit of a photoelectric converter according to an embodiment of the present disclosure is shown. The drive circuit includes:
a first thin film transistor TFT1, where a gate of the first thin film transistor TFT1 is electrically connected to a first electrode, a first electrode of the first thin film transistor TFT1 is electrically connected to a first constant voltage source VDD, the first thin film transistor TFT1 is used for amplifying an electrical signal output from a photoelectric converter 1 and outputting the amplified electrical signal by means of a second electrode of the first thin film transistor TFT1, and the other end of the photoelectric converter 1 is electrically connected to a second constant voltage source Vbias;
a second thin film transistor TFT2, where a first electrode of the second thin film transistor TFT2 is electrically connected to the second electrode of the first thin film transistor TFT1, the second thin film transistor TFT2 is used for reading the amplified electrical signal from the second electrode of the first thin film transistor TFT1 according to a read signal Vread received by a gate of the second thin film transistor TFT2; and
a third thin film transistor TFT3 connected between the first electrode and the first constant voltage source VDD and is used for resetting an electrical signal according to a reset signal Vrst received by a gate of the third thin film transistor TFT3.

For example, in FIG. 22, an electrical signal output by the photoelectric converter 1 after exposure is first amplified by the first thin film transistor TFT1, then the amplified electrical signal is read from the first thin film transistor TFT1 by the second thin film transistor TFT2 according to a read signal Vread to form a data signal Vdata, and finally the photoelectric converter 1 is reset by the third thin film transistor TFT3 according to a received reset signal Vrst.

On the basis of the same inventive concept, an embodiment of the present disclosure provides a detection device. The detection device includes the above detection substrate.

The detection device may be an X-ray flat panel detector or a fingerprint identification device.

Although preferred embodiments of the present disclosure have been described, those skilled in the art can make additional changes and modifications to these embodiments once they learn the basic inventive concept. Therefore, the appended claims are intended to be interpreted as including preferred embodiments and all changes and modifications falling within the scope of the present disclosure.

Apparently, those skilled in the art can make various modifications and variations to the present disclosure without departing from the spirit and scope of the present disclosure. In this way, in the case that these modifications and variations of the present disclosure fall within the scope of the claims of the present disclosure and equivalent technologies thereof, the present disclosure is further intended to include these modifications and variations.

1. A detection substrate, comprising:
a plurality of photoelectric converters arranged in an array, wherein the photoelectric converter comprises a plurality of film layers, an area of an overlapping region between electrode film layers forming an internal capacitor in the photoelectric converter is less than an area of other film layers in the photoelectric converter; and
a drive circuit electrically connected to the photoelectric converters.

2. The detection substrate according to claim 1, wherein the photoelectric converter comprises:
a first electrode layer, a first semiconductor layer, an intrinsic semiconductor layer, a second semiconductor layer and a second electrode layer that are sequentially stacked; the first semiconductor layer and the second semiconductor layer are heavily doped semiconductor layers which are different; and
one electrode film layer of the internal capacitor comprises the first electrode layer and the first semiconductor layer, and the other electrode film layer of the internal capacitor comprises the second electrode layer and the second semiconductor layer.

3. The detection substrate according to claim 2, wherein the first semiconductor layer and the second semiconductor layer are doped with different elements.

4. The detection substrate according to claim 2, wherein an area of an orthographic projection of the second electrode layer and the second semiconductor layer on the intrinsic semiconductor layer is greater than and covers an area of an orthographic projection of the first electrode layer and the first semiconductor layer on the intrinsic semiconductor layer; or
an area of an orthographic projection of the first electrode layer and the first semiconductor layer on the intrinsic semiconductor layer is greater than and covers an area of an orthographic projection of the second electrode layer and the second semiconductor layer on the intrinsic semiconductor layer.

5. The detection substrate according to claim 4, wherein the second electrode layer and the second semiconductor layer have patterns that coincide with each other; and
the first electrode layer and the first semiconductor layer have patterns that coincide with each other.

6. The detection substrate according to claim 1, wherein a pattern of the overlapping region comprises a symmetrical pattern.

7. The detection substrate according to claim 6, wherein the symmetrical pattern is a cross, a center of the cross substantially coincides with a center of the intrinsic semiconductor layer, and the cross extends to an edge of the intrinsic semiconductor layer.

8. The detection substrate according to claim 6, wherein the symmetrical pattern is a ring, the ring is consistent with a shape of an outer contour of the intrinsic semiconductor layer, and the ring is arranged within the outer contour of the intrinsic semiconductor layer and keeps a certain distance from an outer edge of the intrinsic semiconductor layer.

9. The detection substrate according to claim 6, wherein the symmetrical pattern is a plurality of rectangles arranged in an array, and the symmetrical pattern is arranged at corners of the intrinsic semiconductor layer.

10. The detection substrate according to claim 6, wherein the symmetrical pattern is a circle or a rectangle, and a center of the circle or the rectangle substantially coincides with a center of the intrinsic semiconductor layer.

11. The detection substrate according to claim 6, wherein the symmetrical pattern is a plurality of strips arranged at intervals, and the strips extend to an edge of the intrinsic semiconductor layer.

12. The detection substrate according to claim 6, wherein the symmetrical pattern is a grid, the grid comprises a plurality of strips that cross each other, and each strip extends to an edge of the intrinsic semiconductor layer.

13. The detection substrate according to claim 6, wherein the symmetrical pattern is a plane comprising a plurality of circular hollow pattern arranged in an array.

14. The detection substrate according to claim 1, wherein areas of the overlapping regions between the electrode film layers of the internal capacitors of the photoelectric converters are identical to each other.

15. The detection substrate according to claim 1, further comprising:
a plurality of microlenses corresponding to the photoelectric converters in a one-to-one manner and arranged on light entry sides of the photoelectric converters.

16. A detection device, comprising a detection substrate, wherein the detection substrate comprises:
a plurality of photoelectric converters arranged in an array, wherein the photoelectric converter comprises a plurality of film layers, an area of an overlapping region between electrode film layers forming an internal capacitor in the photoelectric converter is less than an area of other film layers in the photoelectric converter; and
a drive circuit electrically connected to the photoelectric converters.

17. The detection device according to claim 16, wherein the photoelectric converter comprises:
a first electrode layer, a first semiconductor layer, an intrinsic semiconductor layer, a second semiconductor layer and a second electrode layer that are sequentially stacked; the first semiconductor layer and the second semiconductor layer are heavily doped semiconductor layers which are different; and
one electrode film layer of the internal capacitor comprises the first electrode layer and the first semiconductor layer, and the other electrode film layer of the internal capacitor comprises the second electrode layer and the second semiconductor layer.

18. The detection device according to claim 17, wherein the first semiconductor layer and the second semiconductor layer are doped with different elements.

19. The detection device according to claim 17, wherein an area of an orthographic projection of the second electrode layer and the second semiconductor layer on the intrinsic semiconductor layer is greater than and covers an area of an orthographic projection of the first electrode layer and the first semiconductor layer on the intrinsic semiconductor layer; or
an area of an orthographic projection of the first electrode layer and the first semiconductor layer on the intrinsic semiconductor layer is greater than and covers an area of an orthographic projection of the second electrode layer and the second semiconductor layer on the intrinsic semiconductor layer.

20. The detection device according to claim 19, wherein the second electrode layer and the second semiconductor layer have patterns that coincide with each other; and
the first electrode layer and the first semiconductor layer have patterns that coincide with each other.
